(19) United States
(12) Patent Application Publication
(10) Pub. No.: US 2026/0105599 A1
(43) Pub. Date: Apr. 16, 2026

(54) APPARATUS AND METHOD FOR MONITORING DISEASE

(71) Applicants: UNIVERSITY OF SOUTHAMPTON, Southampton Hampshire (GB); UNIVERSITY HOSPITAL SOUTHAMPTON NHS FOUNDATION TRUST, Southampton Hampshire (GB)

(72) Inventors: Tom WILKINSON, Southampton Hampshire (GB); Michael John BENNETT, Southampton Hampshire (GB); Benjamin WELHAM, Southampton Hampshire (GB)

(73) Assignees: UNIVERSITY OF SOUTHAMPTON, Southampton Hampshire (GB); UNIVERSITY HOSPITAL SOUTHAMPTON NHS FOUNDATION TRUST, Southampton Hampshire (GB)

(21) Appl. No.: 19/114,099

(22) PCT Filed: Sep. 19, 2023
(86) PCT No.: PCT/GB2023/052416

§ 371 Date: Mar. 21, 2025

(30) Foreign Application Priority Data
Sep. 21, 2022 (GB) 2213773.1

(51) Int. Cl. G06T 7/00; A61B 6/03; A61B 6/50; A61K 51/10

(52) U.S. Cl. CPC G06T 7/0012; A61B 6/504; A61B 6/507; A61K 51/10; A61B 6/032; A61B 6/037; A61K 2123/00; G06T 2207/10116; G06T 2207/30104; G06T 2207/30204

(57) ABSTRACT

A method for determining a measure of disease in a region of the body of a subject. The method receives imaging data from imaging the region of the body of the subject having data representative of a distribution of a marker within the region administered to the subject prior to the imaging. The marker binds to a biological target related to the disease. The imaging data is processed to obtain a measure of marker signal in the region. The measure of marker signal is corrected for an effect on the marker signal of tissue structure within the region. The measure of the disease is determined for the region using the corrected measure of marker signal. Also provided is an apparatus configured to carry out the method.

### PRIORITY CLAIM

This is a U.S. national stage of application No. PCT/GB2023/052416. Priority is claimed on Great Britian Application No.: GB2213773.1, filed on 21 Sep. 2022, the content of which is incorporated herein by reference.

### FIELD OF INVENTION

The invention relates to medical imaging, in particular for monitoring of disease.

### BACKGROUND OF THE INVENTION

Within the healthcare sector there exists a need for better ways to identify which drug a patient is likely to respond to before beginning treatment. Currently over 50% of disease-modifying agents fail in clinic as patient selection based upon blood biomarkers is sub-optimal. When a patient presents with a disease the doctor may prescribe a particular drug and will have to wait several months to see if it has been effective in controlling the condition. If the drug has not worked, the doctor will prescribe a different one and the process repeats until the best drug is found. This wastes precious time and money in the healthcare sector. As the cost of new drugs continues rise, this becomes increasingly important. In addition, within the pharmaceutical industry sector there exists a need for better ways to stratify subjects for clinical trials of novel drugs such as anti-inflammatory or immunomodulatory treatments.

For example, chronic inflammation is a core process in the pathophysiology of some of the world's leading causes of morbidity and mortality. Cardiovascular and cerebrovascular disease is increasingly becoming accepted as an inflammatory disease and are the leading causes of death worldwide, followed by Chronic Obstructive Pulmonary Disease (COPD), which is also characterized by chronic activation of inflammatory pathways, usually due to repeated inhalation of toxins. Further to this, autoimmune inflammatory disorders such as rheumatoid arthritis and inflammatory bowel disease are also highly prevalent chronic inflammatory diseases responsible for significant morbidity worldwide, particularly in Europe and North America. Chronic inflammatory disease is a significant burden on healthcare services and novel disease modifying therapies are much needed. Outcomes in cardiovascular disease have improved with the introduction of primary and secondary prevention strategies, but the development of further therapeutic interventions is ongoing.

Inflammatory diseases have seen recent advances in therapeutic approaches with the introduction of monoclonal antibody therapy in diseases such as rheumatoid arthritis and Crohn's disease. Anti-TNF-α therapy such as infliximab and other biologics including anti-CD20 (rituximab) and anti-IL-6 (tocilizumab or sarilumab) have been introduced as part of established treatment protocols in rheumatoid arthritis. In lung disease, development of novel therapeutics to treat asthma has provided monoclonal antibody therapy for selected patient groups with clear evidence of benefit but the same advances have not been seen in the search for disease modifying therapy in COPD. Studies into anti-IL-5, anti-IL-5Rα and anti-TNF-α therapy have failed to show significant benefit in the COPD population.

The heterogeneity within the COPD disease group is likely to be a key factor in this apparent lack of efficacy, with the current belief being that particular inflammatory phenotypes or endotypes will be responsive to specific therapies. Similarly, despite anti-TNF-α antibodies being an established therapy in the treatment of rheumatoid arthritis, up to 40% of patients fail to show a long-term response to therapy due to heterogeneity in the population, with no currently available means to predict those who will respond¹ as patient genotyping based upon blood biomarkers or clinical features is sub-optimal. Treatments for inflammatory bowel disease are also often ineffective.

Given that there are currently few therapies and interventions that influence disease outcome for patients with airways disease, as well as difficulty in selecting appropriate therapy for the individual in other inflammatory disease, a better understanding of the complex immune processes involved is required. A key scientific challenge, therefore, is to better understand the mechanisms implicated in these inflammatory pathways and to identify targets for therapy.

Technology is now available that allows immunological imaging of tissue and organs using single photon emission computed tomography (SPECT) and positron emission tomography (PET) techniques. These techniques make use of radiopharmaceuticals such as radiolabelled fluorodeoxyglucose (¹⁸F-FDG) or radiolabelled monoclonal antibodies to visualise processes, cells or cytokines associated with immune responses. These nuclear medicine techniques have previously been used within the field of oncology for identifying, locating and assessing the functional state of various cancers.^{5,6} This form of imaging may allow investigators to visualise active inflammation and pathological processes in a way that was only previously possible with tissue biopsy but, instead, over a much larger area and without the need for invasive procedures.²⁻⁴

Extending the same techniques to imaging of the immune system not only provides an opportunity to investigate disease such as chronic inflammatory processes in vivo, but also may permit the prediction of response to specific therapies, particularly monoclonal antibody therapy. Novel, functional imaging of the immune system could provide a means to uncover new knowledge about these processes and has potential as a tool to predict response to disease modifying therapy.

A number of early studies across a range of chronic inflammatory diseases have been conducted, with some indicating that increased uptake of radiolabelled antibody seen on imaging within inflamed tissue has correlated with either improved response to monoclonal antibody therapy or indications for systemic therapy.⁷⁻⁹ However, these studies have been far from conclusive, and further development is required to develop methods and systems that can reliably identify disease markers such as inflamed tissue and evaluate the efficacy of treatments for the disease.

### SUMMARY OF THE INVENTION

According to a first aspect, there is provided a method for determining a measure of a disease in a region of the body of a subject comprising: receiving imaging data obtained from imaging the region of the body of the subject comprising data representative of a distribution of a marker within the region administered to the subject prior to the imaging, wherein the marker binds to a biological target related to the disease; processing the imaging data to obtain a measure of marker signal in the region; correcting the measure of marker signal for an effect on the marker signal of tissue structure within the region; and determining the measure of the disease for the region using the corrected measure of marker signal.

In vivo molecular imaging offers the potential to assess disease in a non-invasive manner, for example by quantifying the driving inflammatory processes in an individual suffering from a chronic inflammatory disease. Correcting for the effects of tissue structure on the signal removes confounding factors that make it difficult to differentiate signal resulting from inflammation from other factors. This enables a new approach to precision medicine that will enable clinicians to select disease modifying therapies that are effective for individual patients first time without invasive procedures.

Optionally, the corrected measure of marker signal is representative of a level of binding of the marker to tissue within the region. Marker bound to tissue is more likely to represent an amount of the biological target in the tissue, in contrast to marker present in the region for other reasons.

Optionally, the disease is an inflammatory disease, and the biological target is a component of an inflammatory pathway. Markers that bind to components of an inflammatory pathway have been tested and represent a promising and valuable application of this technology.

Optionally, the correcting of the measure of marker signal comprises correcting for marker signal due to vascular flow in the region. Marker is typically administered into a patient's blood. This means significant signal arises from free marker in the blood. In addition, areas with higher perfusion due to greater vascular flow may exhibit higher levels of bound marker simply because more marker is available for tissue to interact with. The signal in the region can therefore be distorted depending on how much vasculature is present. Correcting for this gives a better impression of how much of the biological target is present relative to other parts of the region.

Optionally, the correcting for marker signal due to vascular flow comprises determining a vascular density in the region. Vascular density can be used to estimate the proportion of the signal in the region that is due to free marker in blood, rather than marker bound to the biological target.

Optionally, the determining of the vascular density comprises determining the total volume of blood vessels in the region. This can be used to estimate how much signal needs to be corrected for the total blood flow in the region.

Optionally, the imaging data further comprises data representative of tissue structure in the region, and the determining of the vascular density comprises identifying blood vessels in the region using the data representative of tissue structure. Using another source of structural data, for example from another imaging modality better suited to imaging vasculature, can improve the accuracy of the determination of the vascular density.

Optionally, the data representative of tissue structure is obtained by one or more of low-dose computed tomography, high-resolution computed tomography, multi-slice computed tomography, magnetic resonance imaging, plain radiography, or ultrasound. These imaging modalities may be particularly suited to determining tissue structure within the region.

Optionally, the correcting for marker signal due to vascular flow in the region comprises determining a background marker signal corresponding to marker signal from unbound marker in blood. This allows the level of signal due to free marker in the subject's blood at the time of measurement to be accounted for, and a better measure of relative marker activity obtained.

Optionally, the correcting of the measure of marker signal comprises correcting for tissue density in the region. Variations in tissue density in the region can give a false impression of the level of disease in the tissue. Denser tissue will naturally pick up more marker per unit volume than less dense tissue, even at the same level of disease. This could lead to a false estimate that the tissue is more diseased when it is simply denser. Correcting for this effect allows a more accurate estimate of the level of disease to be obtained.

Optionally, the imaging data further comprises data representative of tissue structure in the region, and the correcting for tissue density comprises identifying sub-regions of reduced tissue density in the region using the data representative of tissue structure. Identifying sub-regions of reduced tissue density can allow for an appropriate correction due to that sub-region.

Optionally, the data representative of tissue structure is obtained by one or more of low-dose computed tomography, high-resolution computed tomography, multi-slice computed tomography, magnetic resonance imaging, plain radiography, and ultrasound. These imaging modalities are particularly suited to determining structural information.

Optionally, the data representative of tissue structure is obtained using x-rays, and a sub-region is identified as a sub-region of reduced tissue density if the attenuation of x-rays in the sub-region is below a predetermined threshold. Reduced attenuation of x-rays is a known method of identifying reduced tissue density, particularly in respiratory settings. Using this approach is therefore convenient for integration with existing systems and processes.

Optionally, the correcting for tissue density comprises correcting the measure of marker signal based on a proportion of tissue in the region having reduced density. This allows the method to account for reduced signal that is not due to a reduced level of disease.

Optionally, the correction of the measure of marker signal based on a proportion of tissue in the region having reduced density is determined using measurements of marker signal in a plurality of individuals having varying proportions of tissue in the region having reduced density. This allows appropriate calibration of how much a change in tissue density affects the marker signal.

Optionally, the measure of marker signal is an average marker signal in the region, optionally a median marker signal. Using an average marker signal can give a more accurate measure of the signal in the region as a whole. A median signal may be less distorted by isolated, extreme values than other types of average.

Optionally, the measure of marker signal is normalised by a background marker signal corresponding to marker signal from unbound marker in blood. Normalising the measure by a background signal gives a better measure of relative marker activity in the region.

Optionally, the background marker signal is identified from marker signal in a major blood vessel, optionally the aortic arch. The signal from a major blood vessel is likely to be dominated by the signal due to unbound marker in blood, and so makes a useful source of background signal. Optionally, the background marker signal is an average marker signal in the major blood vessel, optionally a mean marker signal. Averaging over the blood vessel will give a more representative measure of the background activity.

Optionally, the biological target comprises an immune and/or inflammatory signalling protein, for example a cytokine, a chemokine, a cell surface receptor, or an extra-cellular matrix component. These can all be indicative of disease in the body, and so can be used to determine a measure of disease.

Optionally, the marker comprises a radioactive marker, and/or the marker comprises a monoclonal antibody. Monoclonal antibodies can be engineered to have very specific binding action to target specific components, and radioactive markers can be detected easily using non-invasive imaging techniques.

Optionally, the data representative of the distribution within the region of the marker is obtained using single-photon emission computed tomography, SPECT, positron emission tomography, PET, planar scintigraphy, or magnetic resonance imaging. These techniques are all able to provide suitably-resolved imaging of various types of marker.

Optionally, the region of the body of the subject comprises an organ of the subject, for example one or both lungs of the subject or a joint of the subject. These are regions in which common pathological processes such as inflammation are of particular concern and may be of particular interest in patient diagnosis and evaluation of treatment regimes.

Optionally, the imaging data comprises data representative of a distribution within the region of each of a plurality of markers administered to the subject prior to the imaging, wherein each of the plurality of markers binds to a different biological target; the processing of the imaging data comprises processing the imaging data to obtain a measure of marker signal for each of the plurality of markers; the correcting of the measure of marker signal comprises correcting each of the measures of marker signal; and the determining of the measure of disease uses the plurality of corrected measures of marker signal. By combining multiple markers, different biological targets can be identified. In addition, the efficacy of different treatments targeting different biological targets could be evaluated simultaneously. This improves the speed and convenience of diagnostics for the patient.

Optionally, the data representative of tissue structure and the data representative of the distribution of the marker have different resolutions and/or volume segmentations, are measured with the body of the subject in different states, and/or are measured at different times; and the method further comprises aligning the data representative of tissue structure and the data representative of the distribution of the marker. This can allow for different measurements to be taken in series, allowing breaks for the patient to improve comfort. It can also permit the synthesis of data taken with different apparatuses that may have different specifications.

Optionally, the alignment comprises using a non-rigid registration algorithm. This allows the alignment to take account of deformations in the region of the body, for example due to lung inflation during breathing.

Optionally, the alignment comprises deforming the data representative of tissue structure to match the data representative of the distribution of the marker. Deforming the data representative of the distribution of the marker is more likely to distort the measure of marker signal, and so it is preferable to match the data representative of tissue structure to the data representative of the distribution of the marker, rather than the other way around.

According to a further aspect, there is provided a computer program comprising instructions which, when the program is executed by a computer, causes the computer to carry out the method.

According to a further aspect, there is provided a computer-readable medium comprising instructions which, when executed by a computer, causes the computer to carry out the method.

According to a further aspect, there is provided an apparatus for determining a measure of inflammation in a region of the body of a subject comprising a processor configured to carry out the steps of the method.

### BRIEF DESCRIPTION OF THE DRAWINGS

Embodiments of the invention will now be described, by way of a non-limiting example only, with reference to the accompanying drawings in which corresponding reference symbols indicate corresponding parts, and in which according to some embodiments of the disclosure:

FIG. 1 is a flowchart showing a method for determining a measure of disease;

FIG. 2 is a flowchart showing further detail of correcting the measure of marker signal;

FIGS. 3a and 3b show alignment of data representative of tissue structure and data representative of the distribution of marker;

FIG. 4 is a detailed flowchart of processing used in a specific implementation;

FIGS. 5a and 5b are boxplots of median counts at different time points;

FIGS. 6a-6e show normalised median counts at different time points;

FIG. 7 shows the difference in vessel density calculated using scans at different time points;

FIGS. 8a and 8b show the effect of vessel density on median normalised counts;

FIGS. 9a-9c show the effect of reduced tissue density on median normalised counts;

FIGS. 10a and 10b are boxplots of median normalised counts corrected for the effect of vascular flow;

FIGS. 11a-11d are boxplots of median normalised counts corrected for the effect of reduced tissue density;

FIGS. 12a and 12b show marker signal in the right lung of two study participants;

FIG. 13 is a flowchart of an example software platform workflow; and

FIGS. 14a and 14b demonstrate a potential implementation of the method as a clinical platform.

### DETAILED DESCRIPTION OF THE DISCLOSED EMBODIMENTS

New biologic treatments are becoming increasingly widely available for a variety of diseases including cancer and inflammatory diseases such as asthma or rheumatoid arthritis. These can be used to treat disease, but also for diagnostic applications such as cancer screening, and identifying tumour types or infectious agents without the need for an invasive biopsy.

Biologic treatments can be very effective, but not every patient will respond to the same treatment in the same way. Biologics such as monoclonal antibodies target specific arms of the pathways driving a disease, which vary in importance from patient to patient. These drugs are expensive and there is a pressing need for ways to determine which biologic is optimum for a particular patient in order to provide an effective treatment first time. As well as reducing the associated health care costs, this will significantly reduce the burden of disease for the patient by ensuring that they get the most effective treatment as quickly as possible.

In-vivo molecular imaging offers the potential to assess the driving biological processes in an individual suffering from a disease such as chronic inflammatory disease. This new approach to precision medicine has the potential to enable clinicians to select disease modifying therapies and to get it right first time without invasive procedures. However, a major limitation to the translation of molecular imaging to the clinic is the high complexity of image analysis required compared to anatomical medical imaging such as X-Ray, MRI or CT¹⁶ and the ability to quantify a biological signal driven by the abundance of biological targets such as an inflammatory cytokine or molecular immune target in an organ.

The present method allows for quantifying, visualising, and understanding the biology of disease in vivo using non-invasive approaches. The method can form the basis of an imaging platform capable of determining activation of specific biological pathways within an organ system. The method also allows the prediction of patient response to therapy in the clinical setting to optimise selection of specific therapies that an individual will respond to.

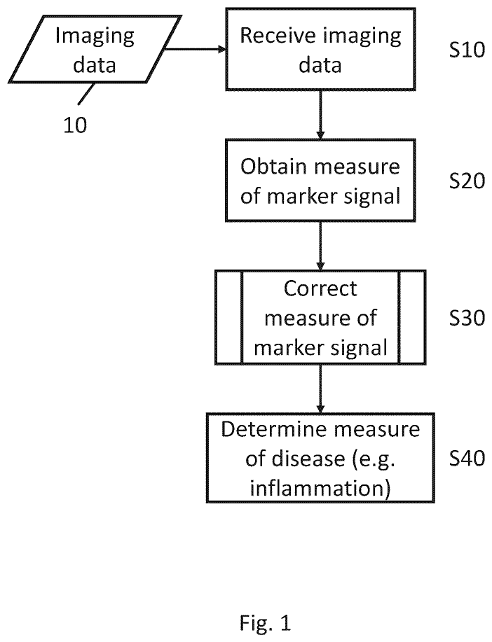

FIG. 1 is a flowchart showing a method for determining a measure of disease in a region of the body of a subject. For example, the disease may be an inflammatory disease, and the measure a measure of inflammation. Alternatively, the disease may be infection, cancer, or a particular type of cancer.

The method comprises receiving S10 imaging data 10 obtained from imaging the region of the body of the subject. The region may be any region of interest where a level of disease is to be determined, or where the effect of a treatment is to be monitored. For example, the region of the body of the subject may comprise an organ of the subject. This could be, for example one or both lungs of the subject or a joint (for example knee or finger joints) of the subject. Monitoring the lungs may be desirable for the assessment of COPD, while monitoring joints may be desirable for the assessment of rheumatoid arthritis (RA).

The imaging data 10 comprises data representative of a distribution of a marker within the region administered to the subject prior to the imaging.

The data representative of the distribution of the marker within the region may be obtained using any suitable imaging technique that is capable of detecting the marker administered to the subject. For example, the data representative of the distribution within the region of the marker may be obtained using any of single-photon emission computed tomography (SPECT), positron emission tomography (PET), planar scintigraphy, or magnetic resonance imaging (MRI). Combinations of these imaging modalities may also be used, for example SPECT-CT, PET-CT, PET-MRI, SPECT-MRI. These choices are advantageous because they make use of established imaging approaches that will be familiar to clinicians and already available in many settings.

Optionally, and as further discussed below, the imaging data 10 may further comprise data representative of tissue structure in the region. The data representative of tissue structure may be obtained by one or more of low-dose computed tomography, high-resolution computed tomography, multi-slice computed tomography, MRI, plain radiography, ultrasound, and Doppler ultrasound. Any suitable combination of methods may be used to obtain the data representative of tissue structure and to obtain the data representative of the distribution of the marker. The data representative of tissue structure may be used to determine the volume of the region of the body, in particular if the imaging data covers more of the body than the region for which the measure of disease is to be determined.

The choice of marker will depend on the imaging technique used to obtain the data representative of the distribution of the marker. For example, the marker may comprise a radioactive marker (which may also be referred to as a tracer or tracer agent). Where MRI is used the marker may comprise a magnetic marker or an MRI contrast agent

The data representative of the distribution of the marker within the region may comprise measurements for each of a plurality of voxels within the region. For example, where the marker comprises a radioactive marker, the measurements may be activity counts for each voxel within a predetermined time period.

The marker binds to a biological target related to the disease. The biological target may be a component of a biological pathway, such as a biological pathway known to be associated with the disease. For example, where the disease is an inflammatory disease, the biological target may be a component of an inflammatory pathway. Where the disease is cancer, the biological target may be a cancer protein, for example a protein associated with the particular type of cancer to be detected and/or treated. Where the disease is an infection, the biological target may be an infectious agent such as a virus or bacterium.

The biological target may comprise an immune and/or inflammatory signalling protein, for example a cytokine, a chemokine, a cell surface receptor, or an extra-cellular matrix component. One specific example used in the example below is tumour necrosis factor, TNF. TNF-α is a proinflammatory cytokine released by macrophages and airway epithelial cells within the lungs of patients with COPD. Levels are known to be increased in the sputum of patients with COPD¹⁴ and TNF-α is central to a number of the inflammatory features seen such as secretion of matrix metalloproteinases (MMPs) and fibroblast proliferation. TNF-α may also be implicated in the cachexia and weight loss experienced as part of the disease.

The marker may comprise a monoclonal antibody. Monoclonal antibodies are advantageous in binding to very specific targets. For example, when assessing COPD, the marker may comprise an anti-TNF-α monoclonal antibody.

The method comprises processing S20 the imaging data to obtain a measure of marker signal in the region. The measure of marker signal may be a measure representative of the entire region. Where the data representative of the distribution of the marker within the region comprises measurements for a plurality of voxels within the region, obtaining the measure of marker signal may comprise combining the measurements from two or more of the voxels. The measure of marker signal may be an average marker signal in the region, optionally a median marker signal. Obtaining the measure of marker signal may further comprise other standard techniques and corrections as appropriate for the imaging modalities used to obtain the data. For example, correction may be applied for partial volume effects when using an imaging modality where the spatial resolution is lower than or similar to the size of the features being measured. For example, this type of correction may be used where PET or SPECT is used.

The measure of marker signal may be normalised by a background marker signal corresponding to marker signal from unbound marker in blood. Use of target to background ratios has been described in previous literature^{8,9}. This can be used to normalize for biological clearance of the marker when measurements are taken at different times after the marker is administered to the subject, and/or to allow for comparison between different subjects who may have different rates of biological clearance.

The background marker signal may be identified from marker signal in a major blood vessel, optionally the aortic arch. The background marker signal may be determined using voxels that correspond to the major blood vessel in the data representative of the distribution of the marker within the region. If the major blood vessel is not within the region for which the imaging data is obtained, the background marker signal may be determined using other data, but this should be measured as close as possible in time to the imaging data. The background marker signal may be an average marker signal in the major blood vessel, optionally a mean marker signal. The background marker signal may be expressed per unit volume.

The method comprises correcting S30 the measure of marker signal for an effect on the marker signal of tissue structure within the region. The corrected measure of marker signal may be representative of a level of binding of the marker to tissue within the region.

In this way, correction determines ‘tissue-bound’ marker as a meaningful measure of inflammatory activity. The step of correcting S30 the measure of marker signal will be discussed in further detail below.

The method comprises determining S40 the measure of disease for the region using the corrected measure of marker signal. The measure of disease may be representative of a level of disease in the region. For example, where the disease is an inflammatory disease, the measure of disease may be representative of a level of inflammation in the region. The measure of disease may represent an amount of the biological target. The corrected measure of marker signal may be used directly as the measure of disease. Optionally, further processing may be applied to the corrected measure of marker signal to obtain the measure of disease, such as normalisation relative to a normal or average amount of the biological target that may for example be calculated from measurements on healthy subjects.

The correcting S30 of the measure of marker signal may comprise correcting for marker signal due to vascular flow in the region. This allows the method to correct for blood vessel density/perfusion of the lung in the region.

The inventors have found that the number of blood vessels per unit volume (or vascular density) was correlated with signal detected within the region. Higher background vascular flow of marker through tissue in the region can create a higher marker signal in the due to unbound marker in the blood providing additional signal. This additional signal reduces the usefulness of the uncorrected measure of marker signal as a measure of disease. The additional signal increases the measure of marker signal without indicating any higher level of the biological component. In addition, higher levels of available marker can lead to a larger amount of marker binding to tissue locally compared to areas with lower vascular flow, even if the underlying level of disease in the tissue is similar. Correcting for marker signal due to vascular flow therefore allows the method to provide a more accurate measure of disease.

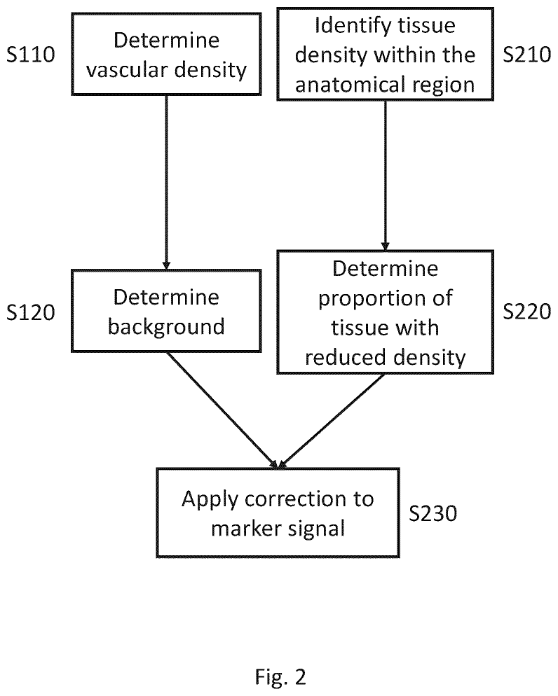

FIG. 2 is a flowchart showing further detail of an implementation of the step of correcting S30 the measure of marker signal for an effect on the marker signal of tissue structure within the region. Steps S110 and S120 correspond to correcting for marker signal due to vascular flow in the region. Steps S210 and S220 correspond to correcting for marker signal due to tissue density in the region. Following the steps S110, S120, S210, and S220, the method comprises applying S230 the correction to the marker signal.

Steps S110 and S120 are shown as being performed alongside steps S210 and S220 in FIG. 2. However, steps S110 and S120 may alternatively be performed before or after steps S210 and S220, or steps S110 and S120 may be omitted entirely. The step S230 of applying the correction to the marker signal follows from both S120 and S220. This reflects the fact that the step S230 of applying the correction to the marker signal may use one or both of the corrections for vascular flow and tissue density, depending on the specific implementation.

In the implementation of FIG. 2, correcting for marker signal due to vascular flow comprises determining S110 a vascular density in the region. Determining S110 the vascular density may comprise determining the total volume of blood vessels in the region. Vascular density may be defined as the total volume of blood vessels in the region divided by the total volume of the region.

The vascular density can be determined by any suitable means based on the imaging data 10 and/or on other sources of data about the patient, for example known or anticipated characteristics of undetected microvasculature. In particular, the imaging data 10 may further comprise data representative of tissue structure in the region. In this case, the determining S110 of the vascular density comprises identifying blood vessels in the region using the data representative of tissue structure.

The blood vessels may be identified using an automated segmentation technique applied to the data representative of tissue structure. The automated segmentation may also be used to determine the volume of the region of the body from the data representative of tissue structure. The automated segmentation technique may be implemented using a machine-learning algorithm. The automated segmentation technique may identify structural features of the tissue, such as blood vessels and fissures in the lung, and may provide a representation of the vascular tree within the region. The automated segmentation technique may make use of prior shape models to detect anatomical regions in particular organs or regions of the body, and/or to aid the identifying of vasculature in the image data 10.

The determining S110 of the vascular density may also take into account data from other sources such as scans of previously resected tissue specimens from the same region of the body (which may be taken from the subject or from other individuals). Such data can provide further information on the predicted microvascular vessel density, which may not be detected in clinical imaging of the subject. These data can be incorporated into the determination of the vascular density, for example by providing an estimate of the typical additional volume of blood vessels that may not be discernible from the imaging data 10 alone.

In the implementation of FIG. 2, correcting for marker signal due to vascular flow in the region further comprises determining S120 a background marker signal corresponding to marker signal from unbound marker in blood. The background marker signal may be obtained as described above, for example using the marker signal in a major blood vessel such as the aortic arch.

Once the background signal and vascular density have been determined, they can be combined to estimate the signal due to unbound marker in vasculature. The proportion of the region occupied by blood vessels can be assumed equal to the vessel density and multiplied by the background signal to determine the expected signal due to vasculature.

In the implementation of FIG. 2, applying S230 the correction to the marker signal comprises subtracting the signal due to vasculature from the measure of marker signal. A specific implementation is provided by the following equation:

$${N}_{T}^{′} = \frac{{M}_{T} - {M}_{Ao} {D}_{v}}{{M}_{Ao}} \qquad ( 1 )$$

where

$${N}_{T}^{′}$$

is the normalised measure of marker signal corrected for vascular flow, M_{AO} is the background signal, Dᵥ is the vessel density in the region of the body, and M_{T} is the measure of the marker signal, which in this case is the average (median) marker signal in the region.

In this case, the background signal M_{AO} is a per-volume value, such that

$${M}_{Ao} = \frac{{R}_{Ao}}{{V}_{Ao}} \qquad ( 2 )$$

where R_{AO} is the raw marker signal within the area used to determine the background (e.g. the aortic arch), and V_{AO} is the volume of the area used to determine the background. As mentioned above, a normalised measure of marker signal N_{T} may be used, which is normalised by the background signal, i.e.

$${N}_{T} = \frac{{M}_{T}}{{M}_{Ao}} .$$

Therefore Eq. (1) can be simplified to normalised by the background signal, i.e.

$${N}_{T}^{′} = {N}_{T} - {D}_{v} \qquad ( 3 )$$

The expression (3) can be explained by considering the hypothetical situation where the entire region was filled with blood and no tissue. If this were the case, the measure of marker signal from the region would be expected to be equal to the background signal, and therefore the normalised measure of marker signal N_{T} would be 1. In this case, the vessel density Dy would also be 1 (because 100% of the region is occupied by blood) and therefore the corrected measure of marker signal would be zero.

The correcting S30 of the measure of marker signal may comprise correcting for tissue density in the region.

In some experiments, presence of emphysema in patients with COPD was identified as a possible factor influencing the measure of marker signal. On this basis, the correcting S30 of the measure of marker signal may comprise correcting for tissue density in the region, such as variations caused by the presence of emphysema. Following investigation, the presence of emphysema was identified as a confounding factor. Strong correlations were found between marker signal and degree of emphysema.

Without wishing to be bound by theory, it is hypothesised that the marker cannot bind as easily or effectively to the biological target in regions of reduced tissue density, and that vascular flow through the tissue is also affected. The corrections for tissue density are thought to represent a composite factor, including loss of small blood vessels, loss of small tissue structures (such as airways and airway epithelium in the lungs), and loss of tissue density. All of these factors are likely to be a medium for marker signal, either due to presence of biological target within the tissue or due to background movement of the marker (for example, marker diffusing into tissue).

As mentioned above, steps S210 and S220 in FIG. 2 correspond to correcting for marker signal due to vascular flow in the region. Steps S210 and S220 are shown as being performed alongside steps S110 and S120 in FIG. 2. However, as discussed for the steps S110 and S120 above, steps S210 and S220 may alternatively be performed before or after steps S110 and S120, or steps S210 and S220 may be omitted entirely. The step S230 of applying the correction to the marker signal may use one or both of the corrections for vascular flow and tissue density, depending on the specific implementation.

In the implementation of FIG. 2, the imaging data 10 further comprises data representative of tissue structure in the region, and the correcting for tissue density comprises identifying S210 sub-regions of reduced tissue density in the region using the data representative of tissue structure. The data representative of tissue structure may be any suitable data as discussed above.

For example, where the data representative of tissue structure is obtained using x-rays, a sub-region may be identified as a sub-region of reduced tissue density if the attenuation of x-rays in the sub-region is below a predetermined threshold. The predetermined threshold may be chosen based on typical attenuation for healthy tissue in the region of the body, for example determined from measurements of other healthy subjects.

In the implementation of FIG. 2, the correcting for tissue density comprises correcting the measure of marker signal based on a proportion of tissue in the region having reduced density. This is achieved by determining S220 the proportion of tissue in the region having reduced density and correcting the measure of marker signal based on the proportion. In the case of emphysema, a commonly-used measure for the proportion of tissue in the region having reduced density is the percentage low attenuation area at −950 Hounsfield Units (% LAA₋₉₅₀).

In the implementation of FIG. 2, applying S230 the correction to the marker signal comprises adding to the measure of marker signal a correction proportional to the proportion of tissue in the region having reduced density. A specific implementation is provided by the following equation:

$${N}_{T}^{″} = {N}_{T} + {mP}_{ld} \qquad ( 4 )$$

where

$${N}_{T}^{″}$$

is the normalised measure of marker signal corrected for tissue density, N_{T} is the normalised measure of marker signal, P_{ld} is the proportion of tissue in the region having reduced density, and m is a correction factor. This allows the correction of the median normalised counts to the level expected if all tissue was normal (i.e. P_{ld}=0).

The correction of the measure of marker signal based on a proportion of tissue in the region having reduced density may be determined using measurements of marker signal in a plurality of individuals having varying proportions of tissue in the region having reduced density. The correction factor may be determined by fitting (e.g. using linear regression) the (uncorrected, but optionally normalised) measure of marker signal as a function of the proportion of tissue in the region having reduced density. For example, using linear regression on the normalised measure of marker signal will result in a linear fit of the form:

$${N}_{T} = b + {aP}_{ld} . \qquad ( 5 )$$

The coefficient α can then be used for the correction factor m in Eq. (4) above.

Different correction factors could be applied depending on what measurements from the plurality of individuals are used to obtain the correction factor. In particular, the time at which the measurements are taken after administration of the marker to the individuals can affect the correction factor due to the change in the level of unbound marker in the subject's blood.

Measurements taken at a later time point (e.g. 24 hours after administration) are generally preferred for determining the correction factor, because this represents a time-point where the marker is more likely to be bound to the biological target or at least evenly distributed throughout the region of the body. However, measurements taken at an earlier time (e.g. 6 hours after administration) may be preferred in some situations because the higher level of unbound marker in blood may allow for more accurate background correction

As discussed above, the method may use imaging data 10 comprising data representative of tissue structure in addition to the data representative of the distribution of the marker. In this case, it is possible that different imaging techniques or modalities may be necessary to acquire the data representative of tissue structure and the data representative of the distribution of the marker. In addition, the scans used to acquire the imaging data 10 are often time-consuming and it may be necessary to provide breaks for the subjects between scan that acquire different parts of the imaging data 10.

For these reasons, it is possible that the data representative of tissue structure and the data representative of the distribution of the marker have different resolutions and/or volume segmentations, are measured with the body of the subject in different states, and/or are measured at different times. In this case, the method may further comprise aligning the data representative of tissue structure and the data representative of the distribution of the marker. This can allow the method to account for factors such as subject positioning relative to the imaging apparatus (e.g. if the subject stands or moves around between scans) and organ movement as part of physiological processes (e.g. breathing motion, cardiac motion).

The alignment preferably comprises deforming the data representative of tissue structure to match the data representative of the distribution of the marker. This may be achieved by segmenting the region of the body into voxels in the data representative of tissue structure, and then mapping these onto the data representative of the distribution of the marker. It is preferable to deform the data representative of tissue structure rather than the data representative of the distribution of the marker, because this leads to less distortion of the measure of marker signal. Once the region has been mapped in this way, the marker signal within each region can be quantified to obtain the measure of marker signal and further analysis performed to determine the measure of disease as described above. The alignment may comprise using a non-rigid registration algorithm.

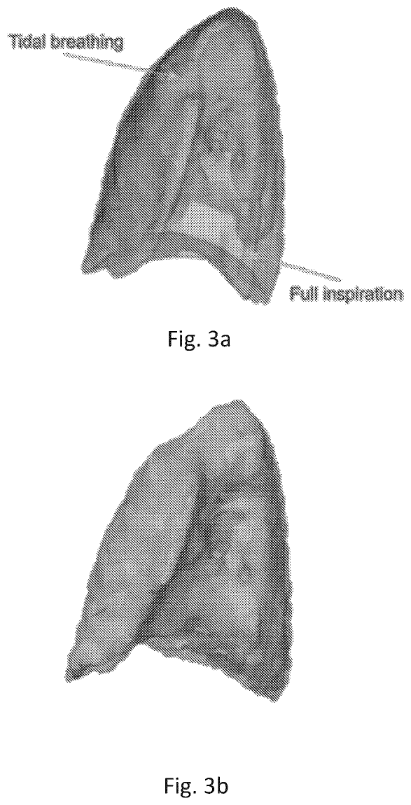

FIG. 3 shows an example of the alignment of two sets of data. FIG. 3a shows two overlapping sets of data for a lung, one taken during tidal breathing of the subject, and the other taken at full inspiration. FIG. 3b shows how the two sets of data are aligned following the application of a non-rigid registration algorithm.

The alignment may comprise other processes, such as downsampling of one of the data representative of tissue structure and the data representative of the distribution of the marker. This may be particularly useful where the resolutions of the two types of data are different.

The imaging data 10 may also include data obtained from contemporaneous tissue samples from the subjects being investigated. For example, where disease in the lungs is being measured, this could be achieved using bronchoscopy and lung biopsy from the subject, or by including patients due to undergo lung resection surgery and undertaking imaging in those participants prior to collecting tissue during surgery for laboratory investigation. These additional data, optionally including other data such as immunohistochemistry detection of the biological target (where this is appropriate for the biological target under consideration) within the same tissue, could be used for calibration of the correction factors used for both vascular flow and tissue density. For example, they could be used to improve estimates of the proportion of tissue having reduced density, or to train the algorithms used for segmentation to identify blood vessels in the region of the body.

Another variation of the method allows for the use of multiple markers simultaneously. In such embodiments, the imaging data comprises data representative of a distribution within the region of each of a plurality of markers administered to the subject prior to the imaging, wherein each of the plurality of markers binds to a different biological target. The processing S20 of the imaging data comprises processing the imaging data to obtain a measure of marker signal for each of the plurality of markers. For example, each of the markers may comprise a different radioisotope, such that the activity from each marker can be separated in the imaging data 10. For example, SPECT-CT is capable of determining activity of different radioisotopes individually by detecting the specific energy of photons emitted from each marker (the process of ‘windowing’ for specific energies) and could therefore detect specific-bound activity of each marker and therefore the most active biological target and/or pathway for that subject.

The correcting S30 of the measure of marker signal comprises correcting each of the measures of marker signal, and the determining S40 of the measure of disease uses the plurality of corrected measures of marker signal.

Determining S40 the measure of disease using the plurality of corrected measures of marker signal may comprise combining the plurality of corrected measures into a single measure of disease. For example, the biological target that exhibits increased activity due to disease may vary between different diseases and/or subjects. Determining a combined measure of disease based on plural corrected measures may therefore provide a better indication of the overall activity of relevant biological pathways, or the specific endotype of the disease.

Determining S40 the measure of disease using the plurality of correct measures may alternatively or additionally comprise determining separate measures of disease for each biological target. The simultaneous use of multiple markers, each binding different biological targets (examples including various cytokines or cell surface receptors) allows the method to detect and quantify the activity of each of the biological targets at the same time. This potentially allows for the assessment of multiple diseases or disease subtypes simultaneously.

This can also allow for the simultaneous assessment of the effectiveness of different therapies in binding to the biological targets, for example if the marker is attached to a therapeutic agent such as a monoclonal antibody. Treatments for diseases can be very effective, but not every patient will respond to the same drug in the same way. This may be due to varying levels of different biological targets, and in particular the specific target targeted by the treatment. Determining the measure of disease using plural different markers binding to different targets can be used to determine which targets should be targeted by treatment for an individual subject.

Another potential application using simultaneous infusion of multiple markers is to investigate non-specific signal from marker which may have diffused into tissue within the region of the body but is not bound to its target component. One of the plurality of markers may be a specific marker targeting the biological target of interest, and another of the plurality of markers could be a ‘negative control’ marker not expected to show specific binding against any target within the region of the body. The negative control marker would provide data on how a marker which is not binding to the biological target behaves in the tissue, and therefore how much of the detected signal from the specific marker is likely to be bound to the biological target.

The method may be carried out by an apparatus for determining a measure of disease in a region of the body of a subject comprising a processor configured to carry out the steps of the method. The apparatus comprises a receiving unit configured to receive imaging data obtained from imaging the region of the body of the subject comprising data representative of a distribution of a marker within the region administered to the subject prior to the imaging, wherein the marker binds to a component of an inflammatory pathway. The apparatus comprises a processing unit configured to process the imaging data to obtain a measure of marker signal in the region. The apparatus comprises a correction unit configured to correct the measure of marker signal for an effect on the marker signal of tissue structure within the region. The apparatus comprises a determining unit configured to determine the measure of inflammation for the region using the corrected measure of marker signal. The apparatus may be a general purpose computer adapted to carry out the method. The configuration of the apparatus may be modified appropriately to correspond to any details of the method discussed above.

### EXAMPLES

The following example demonstrates the applicability of the method to the specific case of determining a measure of inflammation in subjects having COPD using a marker that binds to TNF-α.

### Participant Selection for SPECT-CT Study

Five participants with severe to very severe COPD (as per GOLD criteria) and five participants without any history of underlying lung disease were recruited to the study, undertaken at University Hospital Southampton, United Kingdom. Participants with COPD had a prior clinical diagnosis of COPD, smoking history of >10 pack years and either severe or very severe COPD as per GOLD criteria, with post-bronchodilator FEV1/FVC <0.7 and FEV₁<50% predicted. Healthy volunteers had no history of lung disease and were non-smokers defined as <1 pack-year smoking history. Participant characteristics are outlined in Tables 1-3.

Participants were excluded if they had a history of other respiratory disorders, pneumonia risk factors, chronic kidney disease (CKD) with estimated glomerular filtration rate <60, autoimmune conditions, were taking immunosuppressant medications or other monoclonal antibodies, had a history of adverse reactions to any monoclonal antibody medication, a body mass index (BMI) outside the range 18-30, a urinary catheter in situ or were taking any regular antibacterial, antiviral or respiratory investigational drug within the 30 days prior to enrolment visit. Furthermore, subjects with COPD should not have had an exacerbation requiring treatment with oral corticosteroids within the 30 days prior to enrolment.

After recruitment, all participants underwent pre- and post-bronchodilator spirometry with measurement of diffusion capacity of the lungs for carbon monoxide (DLCO) and lung volumes by body plethysmography. Participants were asked to withhold their usual bronchodilator medication for 12 hours prior to this if applicable. Body measurements and bioelectrical impedance analysis to measure body composition were also undertaken at this visit. Participants underwent blood sampling for safety assessments and TNF-α concentration measurements prior to imaging. Sputum induction for sampling of sputum TNF-α was also undertaken where possible and within COVID-19 infection control policy, both before and after imaging.

TABLE 1

| | | | | | | Pre-bronchodilator | | |
|---|---|---|---|---|---|---|---|---|
| Participant | Group | Age | Gender | Smoking status | Pack years | FEV1 (L) (% predicted) | FVC (L) (% predicted) | $\frac{FEV 1}{FVC}$ |
| 1 | Healthy | 70 | F | Never | 0 | 2.36 (117) | 3.36 (130) | 0.7 |
| 2 | Healthy | 50 | M | Never | 0 | 4.26 (103) | 5.29 (101) | 0.81 |
| 3 | Healthy | 68 | M | Never | 0 | 3.14 (111) | 4.29 (117) | 0.73 |
| 4 | Healthy | 73 | M | Never | 0 | 2.98 (110) | 4.43 (122) | 0.67 |
| 5 | Healthy | 70 | F | Never | 0 | 2.44 (132) | 3.19 (137) | 0.77 |
| 6 | COPD | 75 | M | Ex-smoker | 32 | 1.12 (39) | 2.64 (69) | 0.42 |
| 7 | COPD | 70 | F | Current | 43 | 0.57 (30) | 1.8 (74) | 0.32 |
| 8 | COPD | 74 | M | Ex-smoker | 48 | 0.37 (14) | 2.2 (63) | 0.17 |
| 9 | COPD | 71 | F | Current | 45 | 0.82 (41) | 2.04 (78) | 0.4 |
| 10 | COPD | 70 | M | Ex-smoker | 40 | 0.68 (25) | 2.54 (71) | 0.27 |

TABLE 2

| | | Post-bronchodilator | | |
|---|---|---|---|---|
| | | FEV1 (L) | FVC (L) | |
| Participant | Group | (% predicted) | (% predicted) | FEV1/FVC |
| 1 | Healthy | 2.45 (122) | 3.44 (133) | 0.71 |
| 2 | Healthy | 4.3 (104) | 5.16 (98) | 0.83 |
| 3 | Healthy | 3.33 (120) | 2.71 (112) | 0.81 |
| 4 | Healthy | 3.1 (114) | 4.42 (122) | 0.7 |
| 5 | Healthy | 2.57 (139) | 3.15 (136) | 0.81 |
| 6 | COPD | 1.12 (38) | 2.47 (63) | 0.45 |
| 7 | COPD | 0.57 (30) | 1.69 (70) | 0.34 |
| 8 | COPD | 0.51 (20) | 2.7 (77) | 0.24 |
| 9 | COPD | 0.8 (45) | 2.11 (92) | 0.38 |
| 10 | COPD | 0.79 (29) | 2.9 (81) | 0.27 |

TABLE 3

| | | MEF | TLCO | | | | |
|---|---|---|---|---|---|---|---|
| | | 25-75% (L) | (mmol/min/kPa) | RV | | CAT | mMRC |
| Participant | Group | (% predicted) | (% predicted) | (% predicted) | RV/TLC | score | score |
| 1 | Healthy | 1.52 (87) | 6.47 (98) | 1.9 (98) | 0.35 | 0 | 0 |
| 2 | Healthy | 4.48 (122) | 10.34 (95) | 1.73 (77) | 0.26 | 4 | 0 |
| 3 | Healthy | 3.6 (120) | 6.19 (78) | 1.68 (69) | 0.27 | 4 | 0 |
| 4 | Healthy | 1.53 (54) | 10.21 (127) | 2.96 (114) | 0.41 | 3 | 0 |
| 5 | Healthy | 2.7 (111) | 7.97 (127) | 1.97 (105) | 0.38 | 4 | 0 |
| 6 | COPD | 0.37 (17) | 5.61 (65) | 3.05 (112) | 0.51 | 18 | 2 |
| 7 | COPD | 0.2 (12) | 2.68 (43) | 3.88 (207) | 0.69 | 23 | 2 |
| 8 | COPD | 0.19 (7) | 3.04 (39) | 4.97 (191) | 0.66 | 17 | 3 |
| 9 | COPD | 0.3 (13) | 3.68 (57) | 3.52 (173) | 0.61 | 28 | 2 |
| 10 | COPD | 0.18 (6) | 3.27 (42) | 5.48 (220) | 0.67 | 18 | 3 |

Radiolabelling of Anti-TNF-α with ^{99m}Tc

Radiolabelling was undertaken by the radiopharmacy at University Hospital Southampton. Infliximab, an anti-TNF-α monoclonal antibody (Remicade, Janssen Biotech), was labelled with ^{99m}Tc by the direct labelling method as described previously^{10,11}. Anti-TNF-α was first purified through gel filtration chromatography and isolated based on an optical density of 280 nm using a UV spectrophotometer. The antibody was then reduced using a molar excess of 2-mercaptoethanol leaving free thiol groups available for labelling. After 30 minutes incubation with 2-mercaptoethanol, the reduced antibody was isolated using a PD-10 desalting column.

A methylene diphosphate (MDP) bone-scanning kit (Draximage and Polatom) was reconstituted in 5 ml of 0.9% sodium chloride to provide a solution containing 5 mg medronate, 0.34 mg stannous fluoride and 2 mg p-aminobenzoid acid. Technetium-99m was obtained in the form of sodium pertechnetate, eluted from a ⁹⁹Mo/^{99m}Tc generator. 500 mcg of the reduced antibody was added to the solution followed by 450MBq pertechnetate to produce 99mTc-anti-TNF-α.

Determination of radiochemical purity was undertaken by instant thin layer chromatography (iTLC) paper strips using cut and count method. ITLC is the stationary phase, with 0.9% NaCl being the mobile phase. These conditions give the Rf value of the labelled antibody is Rf-0 and impurities Rf-1. Radiochemical purity was maintained at >95% with the exception of one participant, where radiochemical purity was 91% using this approach (Table 4).

TABLE 4

| | | Radiochemical purity |
|---|---|---|
| Date | Participant | test result in % |
| 12 May 2021 | 5 | 99% |
| 28 Apr. 2021 | 4 | 91% |
| 21 Apr. 2021 | 3 | 97% |
| 25 Nov. 2020 | 10 | 98% |
| 28 Oct. 2020 | 9 | 99% |
| 28 Oct. 2020 | 8 | 99% |
| 17 Mar. 2020 | 2 | 99% |
| 11 Feb. 2020 | 7 | 98% |
| 11 Dec. 2019 | 6 | 99% |
| 05 Nov. 2019 | 1 | 96% |

### Dosimetry Studies

Dosimetry was analysed for the first two participants in the study (one from each group). Estimated effective wholebody dose was 1.6-1.7 mSv (1.6-1.9 mSv when scaled to 370 MBq). This remained well below the 3.6 mSv upper limit permitted by the study protocol. Wholebody retention was calculated to fit a biological t_{1/2} of 23.7 hours (4.8 hour effective t_{1/2}) in the healthy group and 19.5 hours (4.6 hour effective t_{1/2}) in the COPD group, most closely matching expected behaviour from an intact antibody of size 150 kDa such as infliximab¹².

### SPECT-CT Imaging and Biodistribution Studies

Imaging was undertaken at 6 hours (+/−60 minutes) and 24 hours (+/−4 hours) after the beginning of the infusion. Participants in both the healthy volunteer and COPD groups underwent whole body planar imaging, SPECT imaging of the chest and low-dose CT of the thorax at each time point. Participants in the COPD group had an additional high-resolution CT (HRCT) of the thorax at the 6-hour time-point. For the first participant in each group, whole body planar imaging was also undertaken at 3 hours (+/−30 minutes) following infusion for additional dosimetry calculations.

All imaging was obtained using a Symbia Intevo Bold (Siemens Healthcare GmbH, Germany) 16-slice double headed gamma camera system. Whole body planar imaging used a 256×1024 matrix and an acquisition speed of 15 cm/min at the 3 hours, 10 cm/min at 6 hours and 5 cm/min at 24 hours. SPECT imaging at 6 hours was undertaken with a 256×256 matrix with 60 views and 20 s per view. SPECT at 24 hours was undertaken with a 256×256 matrix, 60 views and 40 s per view.

SPECT imaging was undertaken with the patient supine and hands above head. The participant remained in this position for CT imaging which was obtained immediately after SPECT imaging. Free breathing low-dose CT (LDCT) was obtained for all participants at both time points. Inspiratory and expiratory HRCT images were acquired at the 6-hour time point for patients with COPD only.

### Image Processing and Analysis

After the imaging had been performed, the images were initially stored using the hospital Picture Archiving and Communications System (PACS) before removal of all personal identifying information and transfer to a workstation for further analysis.

The CT data, both MSCT (multi-slice CT) and low-dose, were initially analysed using the Southampton Pulmonary Radiomics (SPR) software (version beta-e3b7ef8254). This analysis consisted of:
1) Manual identification of a seed point near the top of the trachea;
2) Automated airway segmentation followed by semi-automated editing to ensure adequate segmentation of all necessary branches;
3) Automated segmentation of the left and right lungs;
4) Manual labelling of the airway branches, especially the right and left main bronchi;
5) Semi-automated segmentation of the lobes and fissures;
6) Automated analysis of the lobes to identify regions of emphysema based on the −950 HU threshold;
7) Segmentation of the blood vessels using a fully automated, AI-driven approach.

The corresponding low-dose CT scan was used to allow the operator to manually identify the location of the aortic arch and to place a spherical region of interest (Rol). The size and position of the Rol was manually adjusted in each case to ensure that as much of the volume of the aortic arch as possible was captured.

As the voxel dimensions of the SPECT and low-dose CT scans were different, the regions identified from the low-dose CT scan were downsampled to match the dimensions of the corresponding SPECT scan. These downsampled regions could then be used to directly mask out regions of interest from the SPECT data.

However, the use of these regions (corresponding to the lobes and the whole left and right lungs) was complicated by the fact that these scans were acquired at suspended full inspiration whilst the SPECT scans were acquired at tidal breathing. This resulted in significant differences in the size and shape of the lung between the two scans meaning that it was not possible to use the MSCT regions directly. This problem was solved by employing a non-rigid registration algorithm to align the regions identified from the MSCT scan with the corresponding features identified from the low-dose CT scans. As the low-dose CT scan and the SPECT scan were automatically spatially aligned by the scanner, it could then be assumed that the registered MSCT regions would be aligned with the corresponding regions in the SPECT scans.

The non-rigid registration was performed on each lobe individually to ensure maximum precision of alignment and to save compute resources. The binary mask resulting from the segmentation of each lobe was used to isolate the MSCT voxels corresponding to only that lobe. The volume corresponding to only the isolated lobe was then extracted from the whole MSCT scan yielding a smaller, more manageable volume on which all further processing was performed. Zero-padding amounting to 10 voxels in each dimension was added to the isolated volume. An initial Affine rigid-registration was then computed using a one-plus-one evolutionary optimizer and Mattes mutual information registration metric. Following the initial rigid-registration, non-rigid registration was performed using a demon-based algorithm with accumulated field smoothing, 5 pyramid levels and 100 iterations per level. This process was found to yield excellent alignment between features identified in the MSCT scans and the correspond regions in the low-dose CT and SPECT scans.

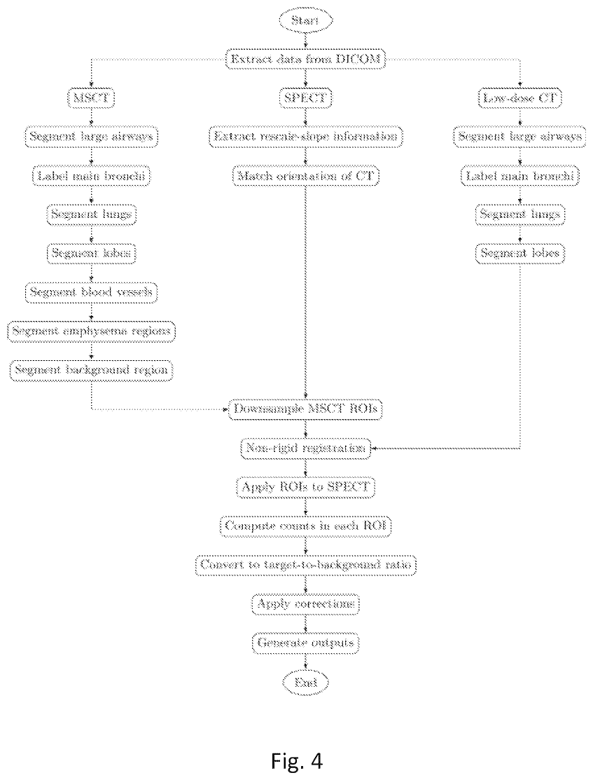

All further processing (including corrections of the measure of marker signal for vascular and tissue density effects) was performed using Matlab (version 2021a, The Mathworks Inc, Natick, MA.) according to the methods described above. A detailed flowchart of the processing used is shown in FIG. 4.

### Statistical Analysis

Median normalised counts were used as the summary statistic for each patient as voxel counts were non-normally distributed. Student's t-test was used for between group comparisons. Pearson's correlation coefficient was used for correlations between median normalised counts and other factors.

### Results

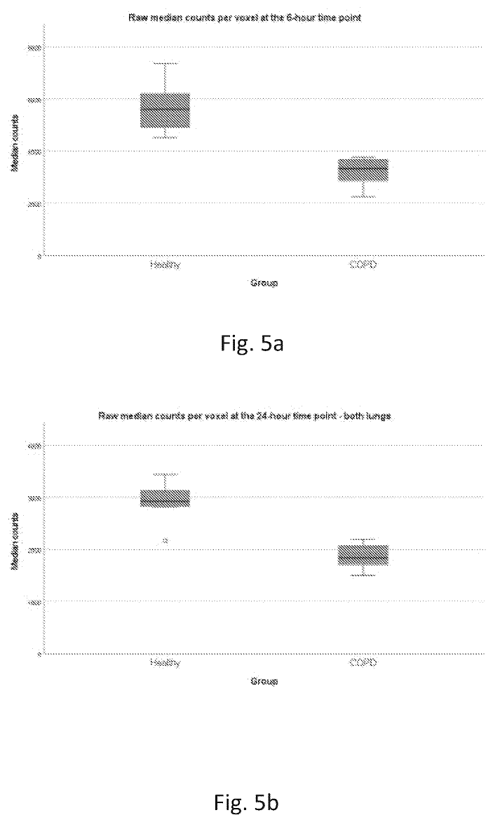

Raw counts were first determined from the SPECT images within the regions of interest defined by the LDCT images taken at the time of the SPECT scan. The raw counts in this example correspond to the measure of marker signal discussed above. Counts were averaged over the entire Rol. The distribution of counts was non-normal over the volumes examined, therefore median counts were used as the summary statistic for each participant. Boxplots representing these results are shown in FIG. 5.

FIG. 5a shows that the mean of these values +/−SD at the 6-hour time point was 5725.0+/−1121.3 in the healthy group and 3182.0+/−631.7 in the COPD group. FIG. 5b shows that at the 24-hour time-point, mean was 2900.0+/−471.5 in the healthy group and 1864.0+/−282.6 in the COPD group. Raw counts were consistently higher at both time points in the healthy group.

The analysis of the MSCT scans yielded much higher detail regions than the corresponding low-dose CT scans. Normalization to ROI area/volume used was applied¹⁵.

The downsampled aortic arch Rol was used to isolate the SPECT voxels corresponding to the blood in the aortic arch. The sum and mean of these voxels values was computed and used as a measurement of the ‘background’ level of activity.

The sum, mean and median values were computed in a similar fashion for each of the downsampled regions identified from the low-dose CT scan (corresponding to the lobes and the whole left and right lungs). In addition, normalized counts were computed by forming the target-to-background (T/B) ratio in order to normalise for biological clearance. This was done by dividing each voxel value by the mean activity identified in the aortic arch. The raw voxel count values were then converted into units of MBq by multiplying the counts value at each voxel by the spatial volume of the voxel and dividing by 1 million.

Normalised counts were calculated on a regional and both-lungs basis at both the 6- and 24-hour time points. Distribution of normalised counts per voxel followed a non-normal distribution, therefore median counts were calculated as the summary statistic for each participant.

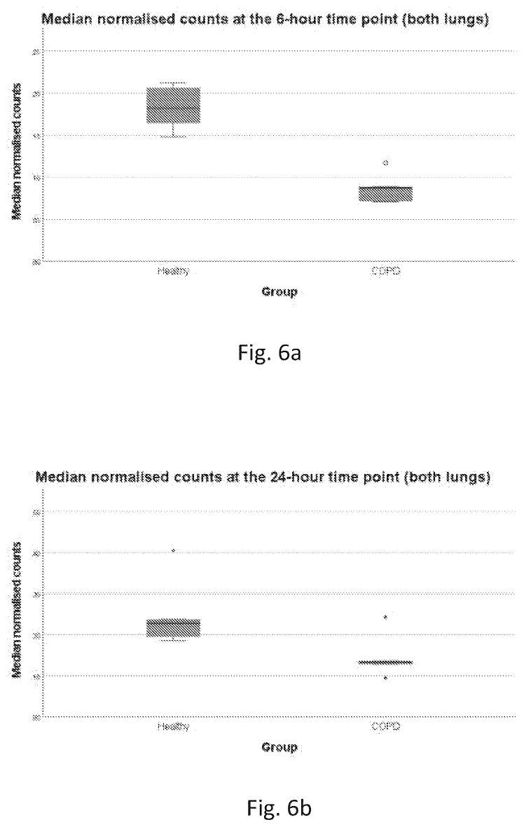

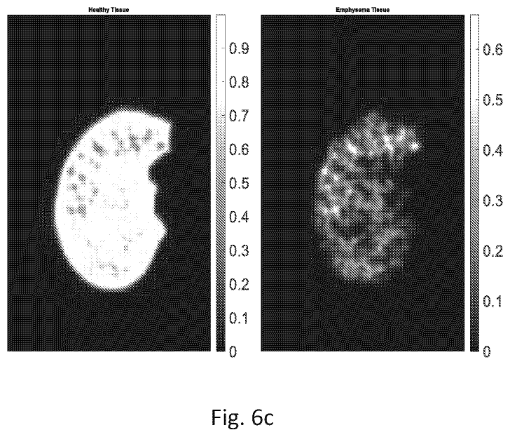

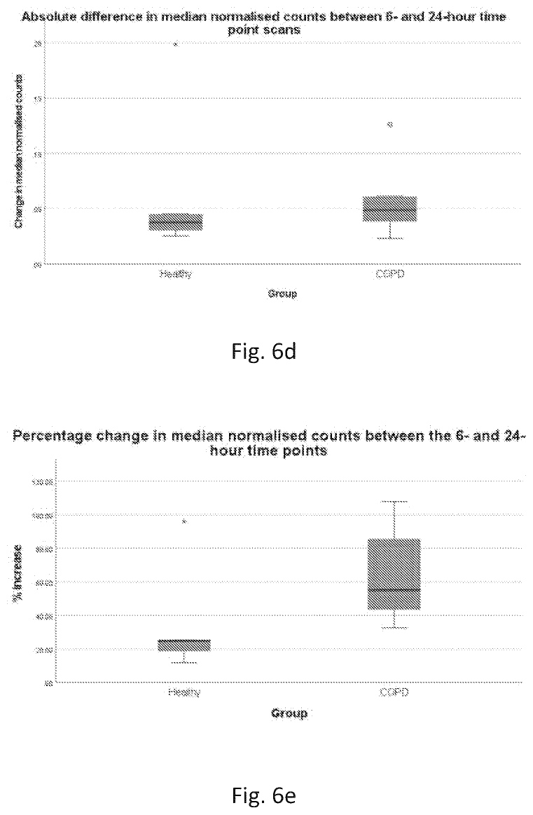

FIG. 6a shows boxplots of the distribution of median normalised counts within the healthy and COPD groups at the 6-hour time point. Within the healthy group, mean of the summary statistics (+/−standard deviation) was 0.182+/−0.027 vs 0.087+/−0.019 in the COPD group.

FIG. 6b shows boxplots of the distribution of median normalised counts within the healthy and COPD groups at the 24-hour (B) time-point. The mean of the summary statistics for the healthy group was 0.250+/−0.089 and 0.147+/−0.056 for the COPD group.

FIG. 6c demonstrates the difference in the normalised counts between a healthy subject (left) and a subject exhibiting COPD (right).

Significant differences were seen between groups, with higher median normalised counts in the healthy group at the 6-hour (p<0.001) but not at the 24-hour time point. The differences seen were due to the factors discussed above such vascular flow and tissue density.

FIG. 6d demonstrates that the absolute difference in detected median normalised counts between the 6- and 24-hour time points did not differ significantly between groups. FIG. 6e shows that the increase in median normalised counts relative to the 6-hour scan was greater in the COPD group, but differences did not reach statistical significance.

The difference in median normalised counts for each participant between the 6- and 24-hour time-point scans was also calculated. Median normalised counts increased at the 24-hour time point in both groups. Absolute difference in median normalised counts +/−SD was 0.067+/−0.073 in the healthy group and 0.059+/−0.040 in the COPD group. The differences between groups were not statistically significant. When the difference in median normalised counts was calculated as a proportion of the 6-hour scan for each patient, a greater increase in median normalised counts was seen in the COPD group. Median normalised counts increased by a mean of 35.38%+/−34.33 in the healthy group and 64.88%+/−31.04 in the COPD group, but the difference between groups did not reach statistical significance.

The higher difference in median normalised counts in the COPD group when calculated as a proportion of the 6-hour scan provided a potential measure of more specific binding to TNF-α at the 24-hour time point in this group.

Previous literature has indicated that small blood vessels detectable on CT are lost with progression both airflow obstruction and emphysema¹³. Variability of vascular density is therefore expected in the population selected for this study.

Vessel density was determined using the automated segmentation techniques described above for the scans taken at the 6-hour and 24-hour time points. Some variation was observed between the vessel density determined using the 6- and 24-hour time-point scans, although this was generally small. The mean+/−SD at the 6-hour time point for both lungs was 0.109+/−0.028 (range 0.069-0.158) and 0.112+/−0.033 (range 0.070-0.171) at the 24-hour time-point.

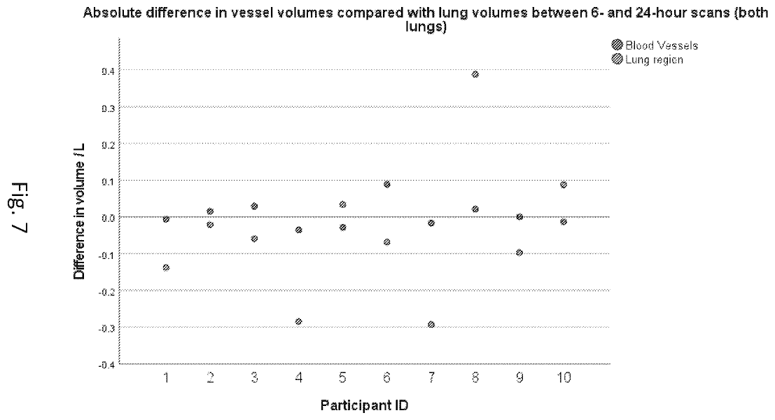

FIG. 7 shows that when the individual components of the calculation were examined, vessel volume differed minimally compared to total CT-measured lung volume, and differences in vessel density between scans were due to changes in overall lung volume.

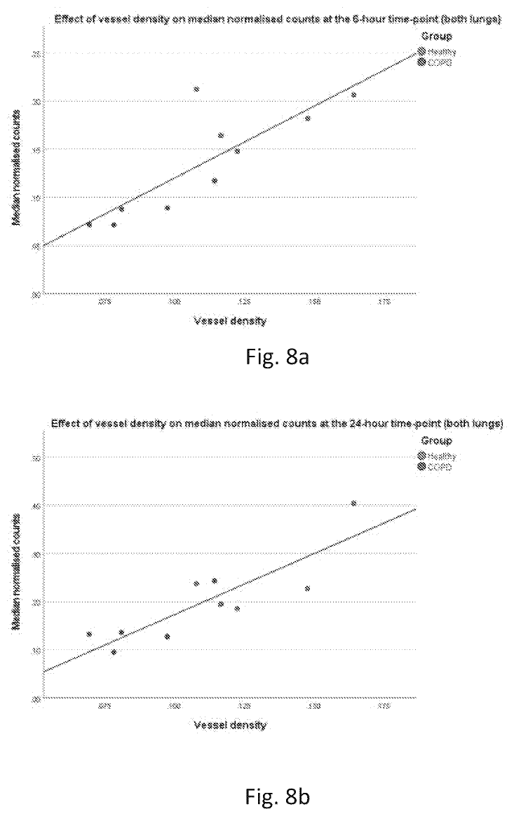

The mean value for CT-detected vessel density was plotted against median normalised counts detected on SPECT to investigate the effect of detectable vascular flow through lung tissue on SPECT activity. FIG. 8a shows the plot for the 6-hour time point, and FIG. 8b shows the plot for the 24-hour time plot. A significant positive correlation was seen at both time-points, with Pearson's correlation coefficient of 0.824 (p=0.003) at the 6-hour time-point and 0.862 (p=0.001) at the 24-hour time-point. This supports the conclusion that vessel density affects the measure of marker signal and supports the validity of the method discussed above.

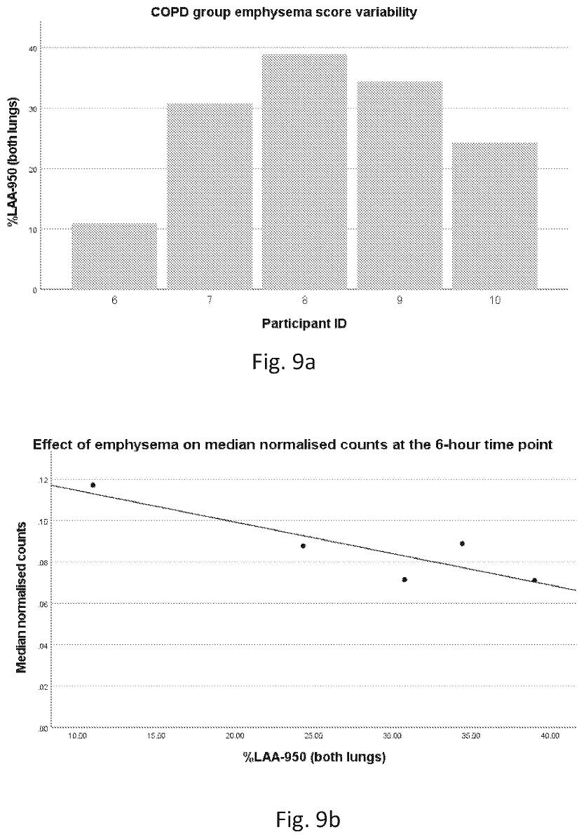

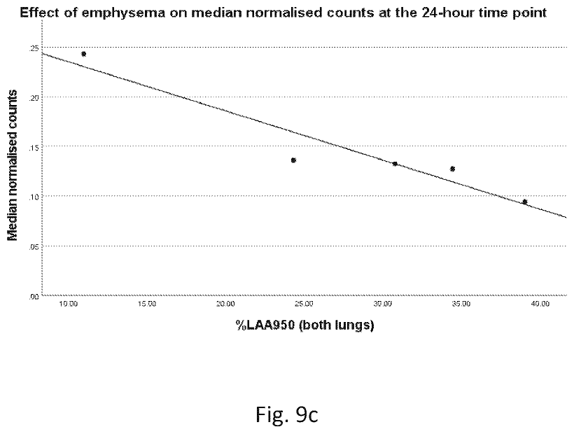

The population selected for the study also varied in the proportion of the region that displayed reduced tissue density. In the specific example, the reduced tissue density is due to emphysema in the patients displaying COPD. FIG. 9a shows the emphysema score variability between subjects with COPD, presented as % LAA₋₉₅₀ (a CT measure of tissue density indicating presence of emphysema) as determined by high-dose inspiratory CT scan.

Values for % LAA₋₉₅₀ were plotted against median normalised counts detected on SPECT. FIG. 9b shows the results for the 6-hour time point, and FIG. 9c shows the results for the 24-hour time point. Clear negative correlations were identified within the HRCT images at both time points. HRCT data was only available for participants with COPD. At the 6-hour time point, Pearson's correlation coefficient was −0.884 (p=0.047) and, at the 24-hour time point, Pearson's r was −0.954 (p=0.012).

The method discussed above was applied to control for the effect of vessel density.

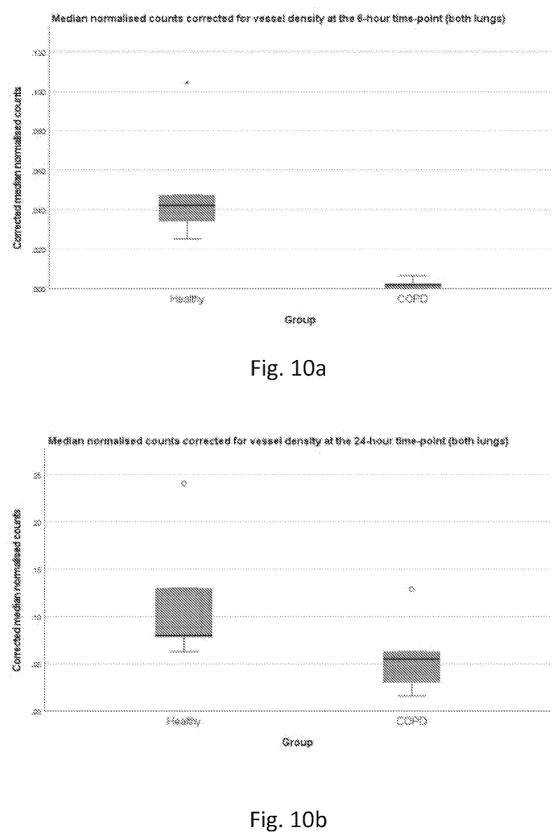

FIG. 10a shows boxplots of median normalised counts after correcting for vascular flow using the method described above at the 6-hour time point. FIG. 10b shows corresponding corrected counts at the 24-hour time point. Participants' median normalised counts were decreased by a mean of 0.110+/−0.030, but uptake remained overall higher in the healthy group compared with the COPD group. A larger effect may be detectable with continued improvements to segmentation algorithms that can detect a higher proportion of the overall vascular tree from the LDCT images.

For correction of effects due to reduced tissue density from emphysema, an analysis of the inspiratory MSCT using a density mask for % LAA₋₉₅₀ for each participant within the COPD group was first undertaken to determine degree of emphysema.

Values for % LAA₋₉₅₀ were determined using a density mask on lung regions identified within the HRCT images. HRCT data was only available for participants with COPD (HRCT was not part of the scan protocol for healthy volunteers). The % LAA₋₉₅₀ results were then plotted against median normalised counts and correlations were drawn by linear regression. The resulting regression coefficient was used to adjust the median normalised counts to a value that would be expected if % LAA₋₉₅₀ was zero, as described above. The effect of this correction method depended on whether the regression line was determined from the results at the 6-hour or the 24-hour time-point.

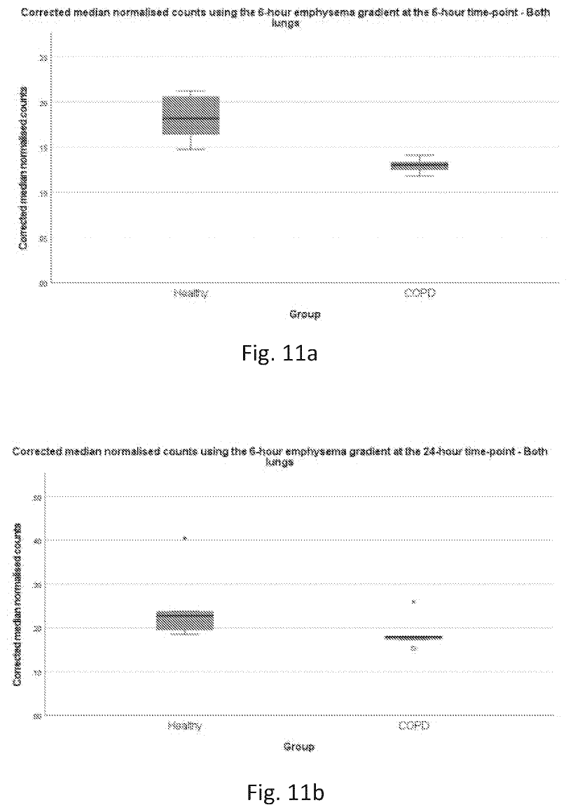

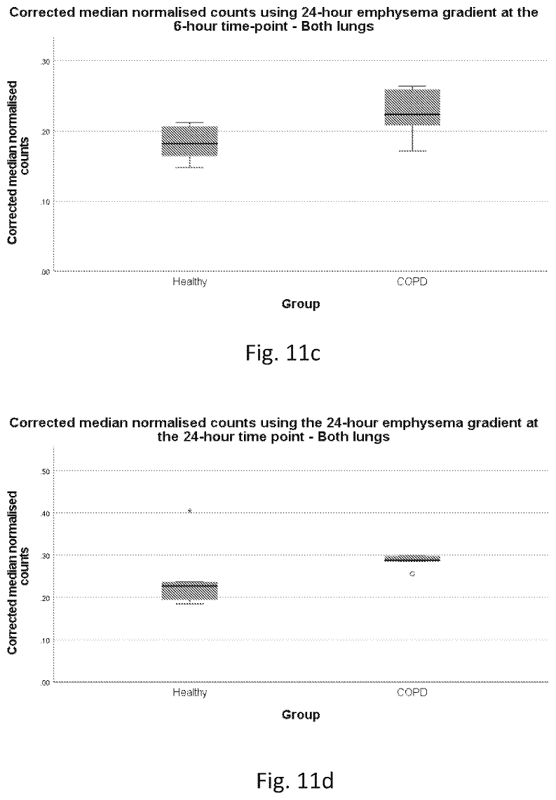

The boxplots in FIG. 11a and FIG. 11b show that the healthy group corrected counts remained higher at both the 6-hour (FIG. 11a) and 24-hour (FIG. 11b) time points if the 6-hour regression line equation was used. Using the 6-hour regression line, corrected counts at the 6-hour time point were (mean+/−SD) 0.182+/−0.027 in the healthy group and 0.129+/−0.009 in the COPD group. At the 24-hour time point this was 0.250+/−0.089 in the healthy group and 0.189+/−0.041 in the COPD group.

However, the boxplots in FIG. 11c and FIG. 11d show that corrected counts were higher in the COPD group at both the 6-hour (FIG. 11c) and 24-hour (FIG. 11d) time-points if the 24-hour regression line was used instead. Using the 24-hour regression line, at the 6-hour time-point corrected counts were 0.182+/−0.027 in the healthy group and 0.225+/−0.038 in the COPD group. At the 24-hour time point this was 0.250+/−0.089 in the healthy group and 0.285+/−0.017 in the COPD group.

### Discussion

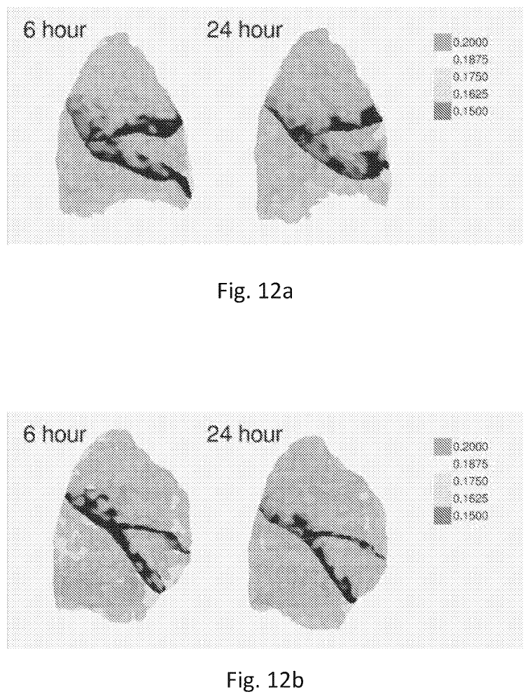

The imaging platform provided evidence of appropriate quantification of signal from the radiopharmaceutical, and this allowed quantitative analysis on a regional and also both lungs basis. FIG. 12 is a three-dimensional representation of the quantification of signal from the radiopharmaceutical in the right lung of two of the study participants at both the 6- and 24-hour time points. FIG. 12a shows subject 1 (healthy) and FIG. 12b shows subject 10 (COPD). Counts were determined on a regional basis, but could also be analysed using median counts over the whole, both-lungs Rol. Variation in density of tissue is visible between groups due to the presence of emphysema in the COPD group.

Results from the study indicate that un-corrected signal is higher in the healthy group, but when corrections are applied for presence of emphysema (as defined by % LAA₋₉₅₀ on HRCT) using an empirical approach, median normalised counts are higher in the COPD group when using the regression line derived from counts detected at the 24-hour time-point for the correction equation.

The biodistribution and clearance in this study corresponded to data from previous studies, with high vascular compartment activity at earlier time points, metabolism and clearance through the hepatic and renal system, as well as uptake in liver and spleen^{8,11}. At later time points, more specific binding to target occurred, with an increase in T/B ratio, as has previously been seen in studies in rheumatoid arthritis⁹ and sarcoidosis⁸.

Further to this, the greater increase in T/B ratio relative to the 6-hour scan within COPD group appears to suggest that more specific binding of the radiopharmaceutical to its target, TNF-α, is seen in these patients. This corresponds with prior literature in patients with sarcoidosis, where a greater percentage change in the T/B ratio was seen in patients with an indication for systemic treatment⁸. A greater increase in absolute T/B values was also seen in responders to anti-TNF-α therapy in a study in rheumatoid arthritis⁹. The more heterogenous nature of lung tissue when compared to synovial tissue may be a reason why this increase in absolute counts was not seen in the present study. Use of the relative change may be more relevant.

Given the central role of TNF-α as a proinflammatory cytokine in COPD, the quantifiable increase in uptake of ^{99m}Tc-infliximab in the current study relative to the 6-hour scan, as well as greater T/B ratio after application of a correction factor would correspond with the expected increased expression of TNF-α in the lungs of patients with COPD.

The study provides evidence that it is possible to image active inflammation within the lungs of patients with COPD using technetium-labelled anti-TNF-α as a marker for inflammation. Previous studies in other disease groups have indicated that uptake can predict response to therapy, indicating that these techniques can be used to evaluate the efficacy of different therapies. With the paucity of disease modifying therapy in COPD, tools to identify responders to therapy are particularly needed for COPD in both the clinical trials setting as well as the clinical setting.

The study provides evidence for the validity of the above-described novel quantitative method for non-invasive detection of target cytokine activity as a measure of active inflammation in the lungs of patients with airways disease. Previous studies have investigated the use of SPECT-CT and scintigraphic approaches for determining target cytokine activity in other inflammatory diseases such as rheumatoid arthritis, Crohn's disease and sarcoidosis^{8,9,11}. This supports the conclusion that the method would also be applicable to other inflammatory diseases and detection of other components of inflammatory pathways.

### Applications

The present method was applied and tested for the lung in the example above, but can be easily applied to other diseases and/or organs, such as an inflammatory disease of the joints such as RA. For example, the present method could be applied to quantification of the activity of components such as IL-5, IL-5Rα and IgE in asthma, or IL-6 in rheumatoid arthritis. Other potential applications include inflammatory diseases of the bowels or liver, or other classes of disease such as cancer or infection.

The present method can be used for various purposes. Because the measure of disease can be determined based on specific biological targets, it can be used to elucidate the underlying active mechanisms involved in the disease. Imaging of disease using the present method could also be used as a biomarker to distinguish specific endotypes or phenotypes within a disease group. For example, the method could be used to help in analysis and identification of cancer subtypes or occult infection. The present method can provide initial stratification of study subjects and generation of the final results in clinical trials, for example aiming to select subgroups of patients with a specific disease who are most likely to respond to the investigational medicinal product (IMP).

The present method could also predict for an individual subject, as discussed above, the probability of response to specific therapy based on the levels of the biological target which the therapy targets. This provides a precision medicine approach to determine the optimal therapy for the patient, and can rapidly identify the best drug for a particular patient. This allows the doctor to get it right first time, reducing time and money spent trying different treatments to find one which is effective for a particular subject.

The method will also contribute to research into new pharmaceuticals and their application to other disease areas by providing a means to rapidly quantify the impact of the drug on the disease using non-invasive methods. It can also be applied to help stratify participants for research studies, leading to improved efficiency and quicker results. This could be extremely valuable for disease groups such as COPD, where heterogeneity in study populations is likely to be the reason that no disease modifying therapy has proven successful so far in clinical trials.

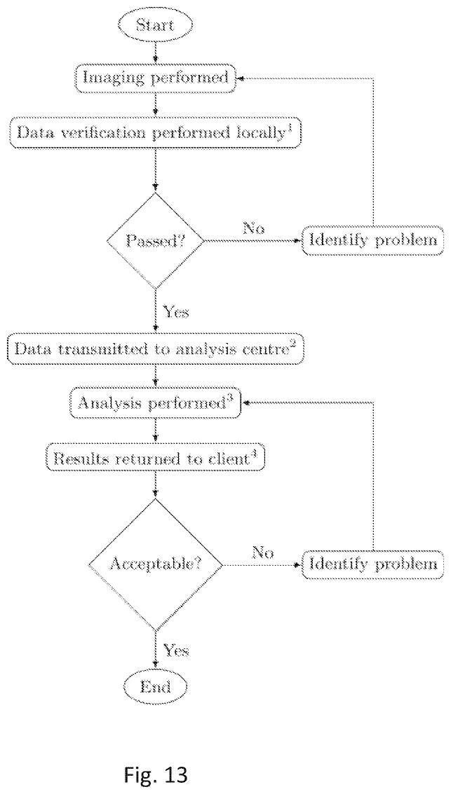

The method may be implemented as part of a platform deployment through a software as a service (SaaS) model that would augment current imaging capabilities. The method could eventually be developed into a clinical imaging platform integrated into existing imaging processes, which is advantageous because the analysis required for this process can be automated. The software platform may interface with hospital computer systems to access imaging data 10 and present results back to the clinician in to help guide treatment. From the patient's perspective, they would only have to attend what to them would seem to be a routine imaging appointment at their local hospital. Some parts of the platform may be provided locally to the hospital while others may be provided remotely. For example, as illustrated in the example flowchart of FIG. 13, basic data verification of the imaging data 10 may be carried out locally such as when the imaging is performed. The analysis to determine the measure of disease may be carried out by a remote system. The division of processes may be chosen based on the availability of computing resources, as well as other considerations such as restrictions due to data protection concerns.

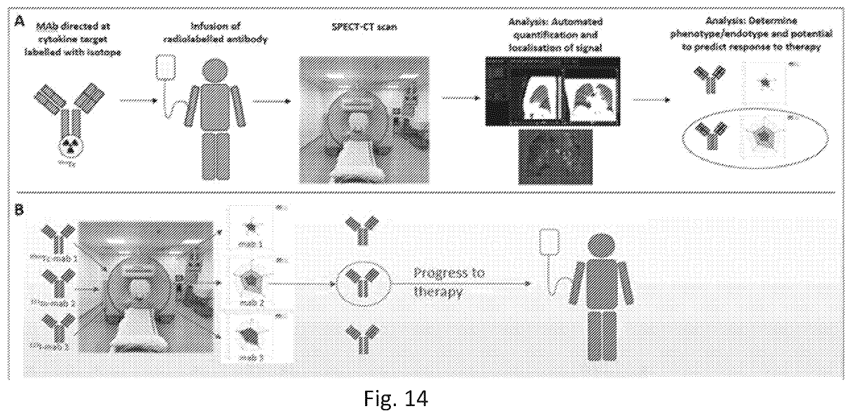

FIG. 14 demonstrates a potential implementation of the method as a clinical platform. FIG. 14a illustrates the overall platform process and aims for a specific biological target, namely a component of an inflammatory pathway for COPD. FIG. 14b shows an anticipated clinical application using a SPECT-CT imaging platform to enable a precision medicine approach to selection of biologic therapy. Multiple biological pathways could be investigated simultaneously with infusion of several monoclonal antibodies each labelled with a different radioisotope. For example, if three monoclonal antibodies were determined to provide potential benefit to a patient, each could be labelled with a different radioisotope and infused simultaneously. Quantification of the uptake of each would lead to identification of the pathway with highest activity to allow the most effective therapy to be selected for the patient.

### REFERENCES

1. Bek, S. et al. Systematic review and meta-Analysis: Pharmacogenetics of anti-TNF treatment response in rheumatoid arthritis. Pharmacogenomics J. 17, 403-411 (2017).
2. Jiemy, W. F. et al. Positron emission tomography (PET) and single photon emission computed tomography (SPECT) imaging of macrophages in large vessel vasculitis: Current status and future prospects. Autoimmun. Rev. 17, 715-726 (2018).
3. Jamar, F., Versari, A., Galli, F., Lecouvet, F. & Signore, A. Molecular Imaging of Inflammatory Arthritis and Related Disorders. Semin. Nucl. Med. 48, 277-290 (2018).
4. Jones, H. A., Marino, P. S., Shakur, B. H. & Morrell, N. W. In vivo assessment of lung inflammatory cell activity in patients with COPD and asthma. Eur. Respir. J. 21, 567-573 (2003).
5. Mariani, G. et al. A review on the clinical uses of SPECT/CT. Eur. J. Nucl. Med. Mol. Imaging 37, 1959-1985 (2010).
6. Israel, O. et al. Two decades of SPECT/CT—the coming of age of a technology: An updated review of literature evidence. Eur. J. Nucl. Med. Mol. Imaging 46, 1990-2012 (2019).
7. Pacilio, M., Lauri, C., Prosperi, D., Petitti, A. & Signore, A. New SPECT and PET Radiopharmaceuticals for Imaging Inflammatory Diseases: A Meta-analysis of the Last 10 Years. Semin. Nucl. Med. 48, 261-276 (2018).
8 Vis, R. et al. 99mTc-anti-TNF-α antibody for the imaging of disease activity in pulmonary sarcoidosis. Eur. Respir. J. 47, 1198-1207 (2016).
9. Conti, F. et al. Role of scintigraphy with 99mTc-infliximab in predicting the response of intraarticular infliximab treatment in patients with refractory monoarthritis. Eur. J. Nucl. Med. Mol. Imaging 39, 1339-1347 (2012).
10. Mather, S. J. & Ellison, D. Reduction-mediated technetium-99m labeling of monoclonal antibodies. J. Nucl. Med. 31, 692-697 (1990).
11. D'Alessandria, C. et al. Use of a 99m-Tc labeled anti-TNF alpha monoclonal antibody in Crohn's disease: in vitro and in vivo studies. Q. J. Nucl. Med. Mol. Imaging 51, 334-342 (2007).
12. Valentin, J. Radiation dose to patients from radiopharmaceuticals. Addendum 3 to ICRP Publication 53. ICRP Publication 106. Approved by the Commission in October 2007. Ann. ICRP 38, 1-197 (2008).
13. Matsuoka, S. et al. Quantitative CT Measurement of Cross-sectional Area of Small Pulmonary Vessel in COPD. Acad. Radiol. 17, 93-99 (2010).
14. Keatings, V. M., Collins, P. D., Scott, D. M. & Barnes, P. J. Differences in interleukin-8 and tumor necrosis factor-alpha in induced sputum from patients with chronic obstructive pulmonary disease or asthma. Am. J. Respir. Crit. Care Med. 153, 530-534 (1996).
15. Galli, F., Lanzolla, T., Pietrangeli, V., Malviya, G., Ricci, A., Bruno, P., Ragni, P., Scopinaro, F., Mariotta, S., & Signore, A. (2015). In vivo evaluation of TNF-alpha in the lungs of patients affected by sarcoidosis. BioMed Research International, 2015. https://doi.org/10.1155/2015/401341
16. Dammes, N., & Peer, D. (2020). Monoclonal antibody-based molecular imaging strategies and theranostic opportunities. In Theranostics (Vol. 10, Issue 2, pp. 938-955). Ivyspring International Publisher. https://doi.org/10.7150/thno.37443

1. A method for determining a measure of a disease in a region of the body of a subject comprising:
receiving imaging data obtained from imaging the region of the body of the subject comprising data representative of a distribution of a marker within the region administered to the subject prior to the imaging, wherein the marker binds to a biological target related to the disease;
processing the imaging data to obtain a measure of marker signal in the region;
correcting the measure of marker signal for an effect on the marker signal of tissue structure within the region; and
determining the measure of the disease for the region using the corrected measure of marker signal.

2. The method of claim 1, wherein the corrected measure of marker signal is representative of a level of binding of the marker to tissue within the region.

3. The method of claim 1, wherein the disease is an inflammatory disease, and the biological target is a component of an inflammatory pathway.

4. The method of claim 1, wherein the correcting of the measure of marker signal comprises correcting for marker signal due to vascular flow in the region.

5. The method of claim 4, wherein the correcting for marker signal due to vascular flow comprises determining a vascular density in the region.

6. The method of claim 5, wherein the determining of the vascular density comprises determining the total volume of blood vessels in the region.

7. The method of claim 5, wherein the imaging data further comprises data representative of tissue structure in the region, and the determining of the vascular density comprises identifying blood vessels in the region using the data representative of tissue structure.

8. The method of claim 7, wherein the data representative of tissue structure is obtained by one or more of low-dose computed tomography, high-resolution computed tomography, multi-slice computed tomography, magnetic resonance imaging, plain radiography, and ultrasound.

9. The method of claim 4, wherein the correcting for marker signal due to vascular flow in the region comprises determining a background marker signal corresponding to marker signal from unbound marker in blood.

10. The method of claim 1, wherein the correcting of the measure of marker signal comprises correcting for tissue density in the region.

11. The method of claim 10, wherein the imaging data further comprises data representative of tissue structure in the region, and the correcting for tissue density comprises identifying sub-regions of reduced tissue density in the region using the data representative of tissue structure.

12. The method of claim 11, wherein the data representative of tissue structure is obtained by one or more of low-dose computed tomography, high-resolution computed tomography, multi-slice computed tomography, magnetic resonance imaging, plain radiography, and ultrasound.

13. The method of claim 12, wherein the data representative of tissue structure is obtained using x-rays, and a sub-region is identified as a sub-region of reduced tissue density if the attenuation of x-rays in the sub-region is below a predetermined threshold.

14. The method of claim 10, wherein the correcting for tissue density comprises correcting the measure of marker signal based on a proportion of tissue in the region having reduced density.

15. The method of claim 14, wherein the correction of the measure of marker signal based on a proportion of tissue in the region having reduced density is determined using measurements of marker signal in a plurality of individuals having varying proportions of tissue in the region having reduced density.

16. The method of claim 1, wherein the measure of marker signal is an average marker signal in the region, optionally a median marker signal.

17. The method of claim 1, wherein the measure of marker signal is normalised by a background marker signal corresponding to marker signal from unbound marker in blood.

18. The method of claim 9, wherein the background marker signal is identified from marker signal in a major blood vessel, optionally the aortic arch.

19. The method of claim 18, wherein the background marker signal is an average marker signal in the major blood vessel, optionally a mean marker signal.

20. The method of claim 1, wherein the biological target comprises an immune and/or inflammatory signaling protein, for example a cytokine, a chemokine, a cell surface receptor, or an extra-cellular matrix component.

21. The method of claim 1, wherein the marker comprises one or both of a radioactive marker and a monoclonal antibody.

22. The method of claim 1, wherein the data representative of the distribution within the region of the marker is obtained using single-photon emission computed tomography, SPECT, positron emission tomography, PET, planar scintigraphy, or magnetic resonance imaging.

23. The method of claim 1, wherein the region of the body of the subject comprises an organ of the subject, for example one or both lungs of the subject or a joint of the subject.

24. The method of claim 1, wherein:
the imaging data comprises data representative of a distribution within the region of each of a plurality of markers administered to the subject prior to the imaging, wherein each of the plurality of markers binds to a different biological target;
the processing of the imaging data comprises processing the imaging data to obtain a measure of marker signal for each of the plurality of markers;
the correcting of the measure of marker signal comprises correcting each of the measures of marker signal; and
the determining of the measure of disease uses the plurality of corrected measures of marker signal.

25. The method of claim 7, wherein:
the data representative of tissue structure and the data representative of the distribution of the marker have different resolutions and/or volume segmentations, are measured with the body of the subject in different states, and/or are measured at different times; and
the method further comprises aligning the data representative of tissue structure and the data representative of the distribution of the marker, for example using a non-rigid registration algorithm.

26. (canceled)

27. The method of claim 25, wherein the alignment comprises deforming the data representative of tissue structure to match the data representative of the distribution of the marker.

28. (canceled)

29. A non-transitory computer-readable medium comprising instructions which, when executed by a computer, causes the computer to carry out the method of claim 1.

30. An apparatus for determining a measure of disease in a region of the body of a subject comprising a processor configured to carry out the steps of the method of claim 1.

31. The method of claim 17, wherein the background marker signal is identified from marker signal in a major blood vessel, optionally the aortic arch.

32. The method of claim 11, wherein:
the data representative of tissue structure and the data representative of the distribution of the marker have different resolutions and/or volume segmentations, are measured with the body of the subject in different states, and/or are measured at different times; and
the method further comprises aligning the data representative of tissue structure and the data representative of the distribution of the marker, for example using a non-rigid registration algorithm.
